(19) United States
(12) Patent Application Publication
(10) Pub. No.: US 2021/0393162 A1
(43) Pub. Date: Dec. 23, 2021

(54) Electronic Devices With Improved Aerobic Capacity Detection

(71) Applicant: Apple Inc., Cupertino, CA (US)

(72) Inventors: Britni A. Crocker, Campbell, CA (US); Katherine Niehaus, San Francisco, CA (US); Aditya Sarathy, Santa Clara, CA (US); Asif Khalak, Belmont, CA (US); Allison L. Gilmore, Redwood City, CA (US); James P. Ochs, San Francisco, CA (US); Bharath Narasimha Rao, San Mateo, CA (US); Gabriel A. Quiroz, San Francisco, CA (US); Hui Chen, Los Altos, CA (US); Kyle A. Reed, San Jose, CA (US); William R. Powers, III, San Francisco, CA (US); Maxsim L. Gibiansky, Sunnyvale, CA (US); Paige N. Stanley, San Jose, CA (US); Umamahesh Srinivas, III, Milpitas, CA (US); Karthik Jayaraman Raghuram, Foster City, CA (US); Adeeti V. Ullal, Emerald Hills, CA (US)

(21) Appl. No.: 17/338,529

(22) Filed: Jun. 3, 2021

Related U.S. Application Data
(60) Provisional application No. 63/041,735, filed on Jun. 19, 2020.

(51) Int. Cl. A61B 5/083; A61B 5/0205; A61B 5/024; A61B 5/00; A61B 5/11; A61B 5/318

(52) U.S. Cl. CPC A61B 5/0833; A61B 5/0205; A61B 5/02416; A61B 2562/0219; A61B 5/1112; A61B 5/318; A61B 5/681

(57) ABSTRACT

One or more electronic device may use motion and/or activity sensors to estimate a user's maximum volumetric flow of oxygen, or VO₂ max. In particular, although a correlation between heart rate and VO₂ max may be linear at high heart rate levels, there is not a linear correlation at lower heart rate levels. Therefore, for users without extensive workout data, the motion sensors and activity sensors may be used to determine maximum calories burned by the user, workout data, including heart rate data, and body metric data. Based on these parameters, a personalized relationship between the user's heart rate and oxygen pulse (which is a function of VO₂) may be determined, even with a lack of high intensity workout data. In this way, a maximum heart rate and therefore a VO₂ max value may be approximated for the user.

This application claims the benefit of provisional patent application No. 63/041,735, filed on Jun. 19, 2020, which is hereby incorporated by reference herein in its entirety.

### FIELD

This relates generally to electronic devices, and, more particularly, to electronic devices with health sensor and detection circuitry.

### BACKGROUND

Electronic devices are often worn or carried near a user's body. The devices may include sensors that are capable of detecting health information, such as heart rate, or movement information, such as distance traveled. One standardized test that is used in diagnostic, clinical settings is based on a user's volumetric flow of oxygen within the user's body, which is commonly referred to as the user's VO₂ and may be measured in liters of Oxygen per minute (L/min). In particular, the maximum value of VO₂ (VO₂ max) for a given user may provide an accurate assessment of the user's health and may provide a high indicator of the user's mortality. However, in clinical settings, a user must run at peak exertion and breathe into a mask that will measure the amount of air used. As a result, many users do not get tested in clinical settings.

Portable electronic devices, such as wearable devices, may have heart rate sensors, motion sensors, and other health sensors that may produce health data. Specifically, these devices may use the user's heart rate during very brisk walking and running workouts to estimate their VO₂ max. Typically, however, these tests require that the user reach approximately 60-70% of their maximal heart rate and that the user run or walk under fairly specific conditions. Therefore, these tests may be triggered when a user manually begins a workout. However, users that work out less frequently and who may have lower fitness levels than users who work out regularly, may not meet the criteria needed to perform a VO₂ max test. Therefore, it may be desirable to estimate a user's VO₂ max in anomalous conditions and/or when a user is not working out (e.g., when a user is walking at a slow pace).

### SUMMARY

Electronic devices such as cellular telephone, wristwatches, and other portable devices are often worn or carried by users. The electronic devices may include motion sensors, such as accelerometers, gyroscopes, and/or global positioning system (GPS) sensors, as examples, that may indicate movement of the electronic device. Additionally, the devices may include health sensors, such as heart rate sensors, electrocardiogram sensors, and/or perspiration sensors, as examples, that may indicate activity information of the user.

To estimate a user's maximum volumetric flow of oxygen, or VO₂ max, control circuitry within the electronic devices may rely on both the movement of the electronic device and the activity information of the user. In particular, the control circuitry may determine a user's calorie data, workout data, and body metrics based on the movement of the device and the activity information, such as the heart rate of the user. The control circuitry may filter at least some of this data to ensure data quality. Moreover, the control circuitry may normalize the heart rate data of the user to account for differences between the user's baseline measurements and anticipated heart rate measurements of similar people in society at large.

The control circuitry may then compute clusters of the calorie data, workout data, and body metrics and aggregate clusters from different periods of time to determine a relationship between the user's heart rate and VO₂. Additionally, the circuitry may perform a probabilistic prior calculation based on the user's age and other factors to determine a predicted relationship between the user's heart rate and VO₂. The two predicted relationships may be combined, and the combined relationship may be projected to the user's estimated maximum heart rate to estimate the user's VO₂ max.

### BRIEF DESCRIPTION OF THE DRAWINGS

FIG. 1 is a drawing of an illustrative wearable electronic device in accordance with an embodiment.

FIG. 2 is a drawing of an illustrative portable device in accordance with an embodiment.

FIG. 3 is a diagram of an illustrative system of two electronic devices in communication with one another in accordance with an embodiment.

FIG. 4 is a diagram of an illustrative motion sensor apparatus and associated circuitry in accordance with an embodiment.

FIG. 5 is a diagram of an illustrative activity sensor apparatus and associated circuitry in accordance with an embodiment.

FIG. 6 is a flowchart of illustrative steps that may be used to calculate a user's VO₂ max in varying conditions.

FIG. 7 is a diagram of illustrative components used by control circuitry to estimate a user's VO₂ max in accordance with an embodiment.

FIG. 8 is a graph of an illustrative relationship between a user's estimated heart rate exertion and the user's actual heart rate exertion in accordance with an embodiment.

FIGS. 9A and 9B are graphs of respective illustrative relationships between heart rate and VO₂, and normalized heart rate and oxygen pulse in accordance with an embodiment.

FIG. 10 is a graph of a range of illustrative relationships between normalized heart rate and oxygen pulse in accordance with an embodiment.

FIG. 11 is a flowchart of illustrative steps used to estimate a user's VO₂ max in accordance with an embodiment.

FIG. 12 is a flowchart of illustrative steps that may be used to conserve device battery while performing low-intensity VO₂ max measurements.

### DETAILED DESCRIPTION

Electronic devices are often carried by users as they conduct their daily activities. For example, a user may carry an electronic device while walking, exercising, or climbing stairs. To provide a user with fitness tracking functionality and other functions, it may be desirable to monitor a user's activities. For example, sensors in an electronic device may monitor user movement. In an illustrative configuration, a motion sensor such as an accelerometer, an altimeter, and/or other sensors in an electronic device may be used in determining when a user has climbed a flight of stairs or performed other physical activities. The same sensors and/or other sensors within the device may be used to determine whether a user has been active or exercised, and the device may track the user's workouts.

To provide a VO₂ max metric for a user, an electronic device may determine a user's maximum calories burned over a desired interval, workout data from the user, and body metric information using motion sensors, other sensors, and/or manual modes of input. Control circuitry within the device may ensure that the workout and body metric data are of sufficient quality, normalize the user's heart rate data, and then analyze the normalized data with the user's maximum calories burned to estimate the user's VO₂ max. To analyze the data, the control circuitry may determine a personalized approximation curve that relates heart rate to VO₂ based on the user's holistic health factors. As a result, this method may be used for any user, including users who do not record workout data very often or at all.

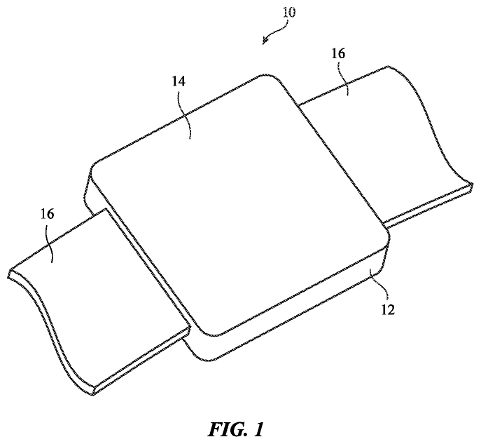

In general, any suitable electronic devices may be used in measuring the user's motion and activity. As shown in FIG. 1, a wearable electronic device 10, which may be a wristwatch device, may have a housing 12, a display 14, and a strap 16. The wristwatch may attach to a user's wrist via strap 16, and provide skin contact on the user's wrist, by which sensors within device 10 may measure signs of physical assertion, such as increased heart rate and perspiration. Additionally, sensors within housing 12 may be used to determine that the wristwatch, and therefore the user, is moving.

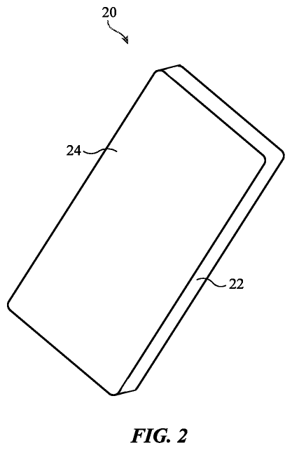

Another illustrative device that may be used to measure the user's motion and activity is shown in FIG. 2. As shown in FIG. 2, a portable device 20, which may be a cellular telephone, for example, has housing 22 and display 24. Sensors within housing 22 may detect motion of the user. In particular, portable device 20 may often be carried in a user's pocket, close to their center of mass, and therefore provide an accurate distance measurement based on movement of the user's legs.

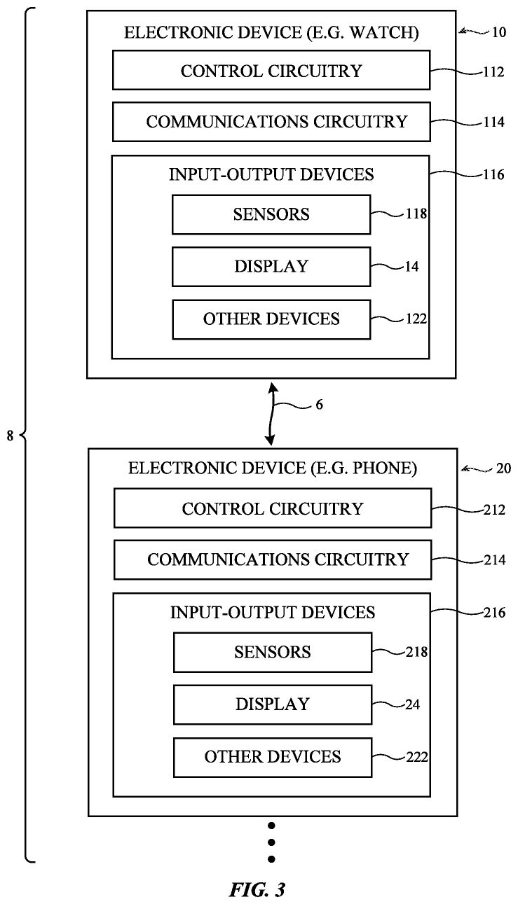

Although electronic devices 10 and 20 may be used separately to determine movement and activity of a user, they may also communicate to provide enhanced measurements. As shown in FIG. 3, electronic device 10 and 20, as well as additional electronic devices may be used in system 8, if desired. Device 10 may be, for example, a wristwatch device as shown in FIG. 1, or may be a cellular telephone, a media player, or other handheld or portable electronic device, a wristband device, a pendant device, a headphone, ear bud, or earpiece device, a head-mounted device such as glasses, goggles, a helmet, or other equipment worn on a user's head, or other wearable or miniature device, a navigation device, or other accessory, and/or equipment that implements the functionality of two or more of these devices. Illustrative configurations in which electronic device 10 is a portable electronic device such as a cellular telephone, wristwatch, or portable computer may sometimes be described herein as an example.

Similarly, electronic device 20, which is illustrated in FIG. 2 to be a cellular telephone, may also be a cellular telephone, a wristwatch, a media player, or other handheld or portable electronic device, a wristband device, a pendant device, a headphone ear bud, or earpiece device, a head-mounted device such as glasses, goggles, a helmet, or other equipment worn on a user's head, or other wearable or miniature device, a navigation device, or other accessory, and/or equipment that implements the functionality of two or more of these devices. Electronic device 20 may communicate with electronic device 10 over path 6. In some embodiments, electronic device 20 may be different from electronic device 10. However, this is merely illustrative. The two electronic devices may be similar if desired. Additionally, electronic devices 10 and 20 may be used together, may be used separately, or may be used in combination with any number of additional electronic devices, as desired.

Additionally, system 8 may include any desired number of electronic devices. Although FIG. 3 shows two electronic devices that communicate over path 6, system 8 may include three or more, four or more, five or more devices. In some embodiments, a single electronic device may be used.

As shown in FIG. 1, electronic devices such as electronic device 10 may have control circuitry 112. Control circuitry 112 may include storage and processing circuitry for controlling the operation of device 10. Circuitry 112 may include storage such as hard disk drive storage, nonvolatile memory (e.g., electrically-programmable-read-only memory configured to form a solid-state drive), volatile memory (e.g., static or dynamic random-access-memory), etc. Processing circuitry in control circuitry 112 may be based on one or more microprocessors, microcontrollers, digital signal processors, baseband processors, power management units, audio chips, graphics processing units, application specific integrated circuits, and other integrated circuits. Software code may be stored on storage in circuitry 112 and run on processing circuitry in circuitry 112 to implement control operations for device 10 (e.g., data gathering operations, operations involving the adjustment of the components of device 10 using control signals, etc.).

Electronic device 10 may include wired and wireless communications circuitry. For example, electronic device 10 may include radio-frequency transceiver circuitry 114 such as cellular telephone transceiver circuitry, wireless local area network transceiver circuitry (e.g., WiFi® circuitry), short-range radio-frequency transceiver circuitry that communicates over short distances using ultra high frequency radio waves (e.g., Bluetooth® circuitry operating at 2.4 GHz or other short-range transceiver circuitry), millimeter wave transceiver circuitry, and/or other wireless communications circuitry.

Device 10 may include input-output devices 116. Input-output devices 116 may be used to allow a user to provide device 10 with user input. Input-output devices 116 may also be used to gather information on the environment in which device 10 is operating. Output components in devices 116 may allow device 10 to provide a user with output and may be used to communicate with external electrical equipment.

In some embodiments, the sensors in one of electronic device 10 and electronic device 20 may be used to calibrate the other device. For example, if electronic device 10 is a wearable electronic device and electronic device 20 is a cellular telephone, the motion sensors within electronic device 20 may provide motion data to the wristwatch, which may calibrate its motion sensors based on the motion data from the telephone. This may be beneficial, as the cellular telephone may be carried in a user's pocket, closer to their center of mass, than on the wrist of the user. However, this is merely illustrative. In general, any number of electronic devices in system 8 may generate data that may be communicated to other devices within system 8 and used to calibrate sensors within those other devices. In this way, the accuracy of the devices in the system may be improved, even when the devices are used individually at a later time.

As shown in FIG. 3, input-output devices 116 may include one or more optional displays such as displays 14. Displays 14 may be organic light-emitting diode displays or other displays with light-emitting diodes, liquid crystal displays, or other displays. Displays 14 may be touch sensitive (e.g., displays 14 may include two-dimensional touch sensors for capturing touch input from a user) and/or displays 14 may be insensitive to touch.

Input-output circuitry 116 may include sensors 118. Sensors 118 may include, for example, three-dimensional sensors (e.g., three-dimensional image sensors such as structured light sensors that emit beams of light and that use two-dimensional digital image sensors to gather image data for three-dimensional images from light spots that are produced when a target is illuminated by the beams of light, binocular three-dimensional image sensors that gather three-dimensional images using two or more cameras in a binocular imaging arrangement, three-dimensional lidar (light detection and ranging) sensors, three-dimensional radio-frequency sensors, or other sensors that gather three-dimensional image data), cameras (e.g., infrared and/or visible digital image sensors), gaze tracking sensors (e.g., a gaze tracking system based on an image sensor and, if desired, a light source that emits one or more beams of light that are tracked using the image sensor after reflecting from a user's eyes), touch sensors, capacitive proximity sensors, light-based (optical) proximity sensors, other proximity sensors, force sensors, sensors such as contact sensors based on switches, gas sensors, pressure sensors, moisture sensors, magnetic sensors (e.g., a magnetometer), audio sensors (microphones), ambient light sensors, microphones for gathering voice commands and other audio input, sensors that are configured to gather information on motion, position, and/or orientation (e.g., accelerometers, gyroscopes, pressure sensors, compasses, and/or inertial measurement units that include all of these sensors or a subset of one or two of these sensors), health sensors that measure various biometric information (e.g., heartrate sensors, such as a photoplethysmography sensor), electrocardiogram sensors, and perspiration sensors) and/or other sensors.

User input and other information may be gathered using sensors and other input devices in input-output devices 116. If desired, input-output devices 116 may include other devices 122 such as haptic output devices (e.g., vibrating components), light-emitting diodes and other light sources, speakers such as ear speakers for producing audio output, circuits for receiving wireless power, circuits for transmitting power wirelessly to other devices, batteries and other energy storage devices (e.g., capacitors), joysticks, buttons, and/or other components.

Similarly, electronic device 20 may have control circuitry 212, communication circuitry 214, and input-output devices 216. Input-output devices 216 may include sensors 218, optional display 24, and other devices 222. Control circuitry 212, communication circuitry 214, input-output devices 216, sensors 218, display 24, and other devices 222 may function similarly as described above in regards to the corresponding parts of electronic device 10. However, electronic device 20 may have different configurations of control circuitry, different bands of communications circuitry, and different combinations of sensors, if desired.

During operation, the communications circuitry of the devices in system 8 (e.g., communications circuitry 112 and communications circuitry 212), may be used to support communication between the electronic devices. For example, one electronic device may transmit video data, audio data, and/or other data to another electronic device in system 8. Bluetooth circuitry may transmit Bluetooth advertising packets and other Bluetooth packets that are received by Bluetooth receivers in nearby devices. Electronic devices in system 8 may use wired and/or wireless communications circuitry to communicate through one or more communications networks (e.g., the internet, local area networks, etc.). The communications circuitry may be used to allow data to be transmitted to and/or received by device 10 from external equipment (e.g., a tethered computer, a portable device such as a handheld device or laptop computer, online computing equipment such as a remote server or other remote computing equipment, an accessory such as a hands-free audio system in a vehicle or a wireless headset, or other electrical equipment) and/or to provide data to external equipment.

During operation, devices 10 and 20 may transmit wireless signals such as Bluetooth signals or other short-range wireless signals and may monitor for these signals from other devices.

For example, devices 10 may transmit Bluetooth signals such as Bluetooth advertising packets that are received by other devices 10. Transmitting devices 10 may sometimes be referred to as remote devices, whereas receiving devices 10 may sometimes be referred to as local devices. In transmitting Bluetooth advertisements (advertisement packets), each remote device may include information in the transmitted advertisements on the recent movement activity of that remote device and other information about the state of the remote device. Movement activity, which may sometimes be referred to as motion context, user motion information, or motion activity information, reflects the recent activities of the user of the remote device involving movement of the user's body (e.g. activities such as resting by sitting and/or standing or moving by walking, running, and/or cycling), and may be shared over Bluetooth between devices. However, any desired protocol may be used to share movement activity between devices in system 8, if desired.

During operation, devices 10 and/or 20 may use sensors 118, wireless circuitry such as satellite navigation system circuitry, and/or other circuitry in making measurements that are used in determining a device's motion context. For example, motion data from an accelerometer and/or an inertial measurement unit may be used to identify if a user's motions (e.g., repetitive up and down motions and/or other motions with a particular intensity, a particular cadence, or other recognizable pattern) correspond to walking, running, or cycling. If desired, location information from a satellite navigation system receiver may be used in determining a user's velocity and thereby determining whether a user is or is not walking, running, or cycling. In some arrangements, the frequency with which a user's cellular telephone transceiver links to different cellular telephone towers may be analyzed to help determine the user's motion. The user's frequency of linking to or receiving signals from different wireless local area network hotspots may also be analyzed to help determine the user's motion and/or other sensor information (e.g., altimeter readings indicating changes in altitude, etc.) may be gathered and processed to determine a user's activity. These techniques and/or other techniques may be used in determining motion context.

In addition to gathering and processing sensor data and other data indicative of the user's motion context, control circuitry 112 in device 10 may, if desired, monitor whether device 10 is wirelessly linked by a short-range wireless link (e.g., via Bluetooth) to handsfree audio systems in vehicles or other vehicle equipment known to be located in or associated with vehicles. In this way, the in-vehicle status of device 10 can be determined. For example, control circuitry 112 in a given device can determine whether the given device is preset in a vehicle or not based on whether circuitry 12 is or is not wirelessly linked with an in-vehicle hands-free system.

In addition to this presence-in-vehicle state information, control circuitry 112 can determine other information about the location of device 10. As an example, control circuitry 112 can conclude that a device is indoors if the device is linked by a short-range wireless link to in-home equipment (e.g., a set-top box, television, countertop speaker, in-home desktop computer, etc.) and can determine that the device is not indoors (and is therefore outdoors) if the device is not linked to this type of in-home equipment and, if desired, sensors in the device sense one or more additional indicators of presence in an outdoors environment such as bright sunlight, etc. In general, any suitable device status information (e.g. device context such as in-vehicle states, indoor-outdoor states, etc.) may be determined by devices 10 and can potentially be shared between devices, as appropriate.

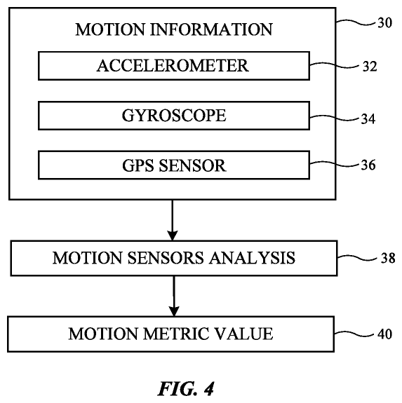

In some embodiments, devices 10 and/or 20 (and/or other devices within system 8) may determine motion of a user. As shown in FIG. 4, motion information 30 may be determined using one or more sensors, such as sensors 118 of device 10 or sensors 218 of device 20. Sensors 118 and/or sensors 218 may include one or more of accelerometer 32, gyroscope 34, and global positioning system (GPS) sensor 36 to measure motion information 30, as examples. Accelerometer 32 may be a two-dimensional or three-dimensional accelerometer (e.g., accelerometer 32 may measure motion in two directions or three directions). In some embodiments, sensors 118/218 may include other motion sensors or other sensors that may be used to detect motion more generally, such as pressure sensors, cameras, light sensors, microphones, or other sensors. However, this is merely illustrative. In general, sensors 118 and/or sensors 218 may include any desired sensors to measure motion of the associated device.

Using data generated by the sensors that collect the motion information, control circuitry, such as control circuitry 112 of device 10, may perform a motion sensor analysis 38 by analyzing the data generated by the one or more sensors. For example, the control circuitry may compare the data generated by each sensor and fuse the data to determine a motion metric value 40. This may be done statistically through weighting, removing outlier measurements from the set, averaging the data, or any other desired method. Motion metric value 40 may be stored within the storage circuitry of the electronic device.

In general, the sensors used to calculate motion metric value 40 may automatically obtain updated motion data at any desired time interval and/or be manually triggered by actions of a user. In either case, the motion metric value 40 may be updated and logged within the storage circuitry when there is enough data to calculate the metric value.

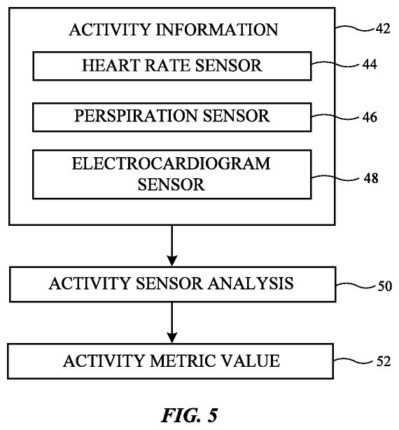

In addition to calculating the motion of the device, sensors with electronic device 10 and/or device 20 may determine activity information of the user. As shown in FIG. 5, activity information 42 may be determined one or more sensors, such as sensors 118 of device 10 or sensors 218 of device 20. Sensors 118 and/or sensors 218 may include one or more of heart rate sensor 44, perspiration sensor 46, and electrocardiogram (EKG) sensor 48 to measure activity information 42, as examples. These sensors may also be used in conjunction with the motion sensors described in connection with FIG. 4, if desired.

Using data generated by the activity information sensors (and the motion information sensors, if desired), control circuitry, such as control circuitry 112 of device 10, may perform an activity sensor analysis 50 by analyzing the data generated by the one or more sensors. For example, the control circuitry may compare the data generated by each sensor and fuse the data to determine an activity metric value 52. This may be done statistically through weighting, removing outlier measurements from the set, averaging the data, or any other desired method. Activity metric value 52 may be stored within the storage circuitry of the electronic device.

In general, the sensors used to calculate activity metric value 52 may automatically obtain updated motion data at any desired time interval and/or be manually triggered by actions of a user. In one example, the electronic device may be placed into an exercise mode, in which the activity information sensors and/or the motion sensors are activated more frequently to determine the user's biometric information more often. In any case, the activity metric value 52 may be updated and logged within the storage circuitry when there is enough data to calculate the metric value.

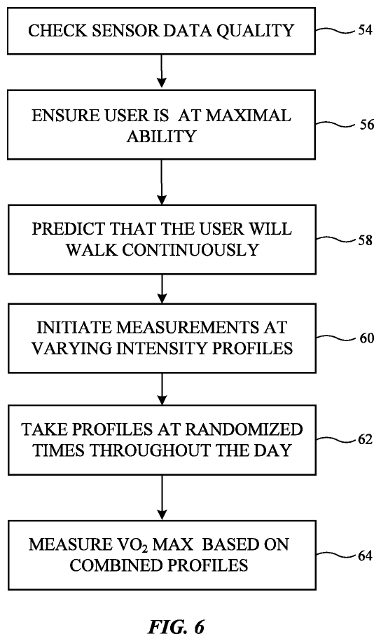

Based on the motion metric value, the activity metric value, and any other desired values, control circuitry within the electronic device, such as control circuitry 112 of device 10, may estimate a VO₂ max value for the user. A flowchart of illustrative steps that may be used to determine the VO₂ max, despite the possibility of conditions that may adversely impact the test, is shown in FIG. 6.

As shown in FIG. 6, at step 54, the control circuitry may check the data obtained from the sensors within the device, such as the motion sensors and the activity sensors. In particular, to accurately determine the VO₂ max value, sensors must have sufficient quality. Therefore, a heart rate sensor and motion sensors may be used, and the data checked. In one example, these sensors may be calibrated regularly (e.g., using data from a second electronic device within system 8 to check and correct sensor readings), and step 54 may be skipped.

At step 56, the system may ensure that the user is in a state that reflects their maximal ability. In particular, the device may use the motion and activity sensors, such as a heart rate sensor and an accelerometer, to determine whether they recently completed a fatiguing exercise by calculating the user's heart rate, calories burned, and step rate or cadence. Additionally or alternatively, the circuitry may compare the recent heart rate to the user's resting heart rate or to the user's typical walking heart rates (which may be regularly calculated and stored within the device's circuitry, if desired). If the user has recently engaged in a fatiguing workout (e.g., their heart rate is over a threshold with respect to their resting heart rate or their typical walking heart rate), the system may postpone the VO₂ max test, as performing the test during this period may lead to erroneous results. On the other hand, if the user has not recently engaged in a fatiguing exercise, the system may proceed to step 58.

At step 58, the circuitry may predict that the user will walk continuously for some period of time. In one example, the system may test for VO₂ max when the user has been continuously for a threshold period of time, and may stop the test when the user stops walking. Alternatively or additionally, the circuitry may determine the user's cadence or calories burned using the motion sensors in the device. For example, the user's cadence or calories burned may be compared to their median or typical cadence or calories burned from the prior week (or any other desired time period). If the user's cadence or calories burned are atypically low, this may indicate atypical walking behaviors, such as walking a dog or walking with a slower person, and the system may postpone the VO₂ max test. On the other hand, if the user's cadence or calories burned are higher than average, the user may intend to walk faster, and it may be desirable to conduct a VO₂ test during that period, and may proceed to step 60.

At step 60, the system may initiate measurements with varied intensity profiles. The system may initiate these measurements using the motion sensors, such as the motion sensors 30 of FIG. 4. For example, the motion sensors may be activated for the varied intensity profiles, may be operated at a higher frequency during the varied intensity profiles, or otherwise be modified when the varied intensity profiles are taken. The motion sensors may include an accelerometer, a barometer (also referred to as a pressure sensor herein), and a GPS sensor, as examples. Using these sensors, the circuitry may initiate measurements of the user's cadence, any incline or hill that the user may be walking on, and/or the speeds at which the user is moving. Additionally, activity sensors, such as a heart rate sensor and/or a perspiration sensor, may also be activated or more frequently used during the varied intensity profiles. Additionally or alternatively, a secondary device, such as a cellular telephone, may also have motion sensors that may add additional motion data that may be used in VO₂ max calculations.

At step 62, the circuitry may ensure that measurements will occur at randomized times throughout the day. In particular, because users may have different activity profiles and behaviors that vary throughout the day, taking randomized readings may correct for abnormalities in the user behavior. In this way, the circuitry may activate the sensors required to perform the VO₂ max test only when certain criteria are met and then ensure that the measurements are conducted using various intensity profiles and at randomized times throughout the day to provide for more accurate VO₂ max estimation. To determine the user's VO₂ max during these selected periods, the circuitry may use predetermined correlations between heart rate and VO₂, along with extrapolating the user's activity to a maximum heart rate, at which point the user's VO₂ max may be approximated.

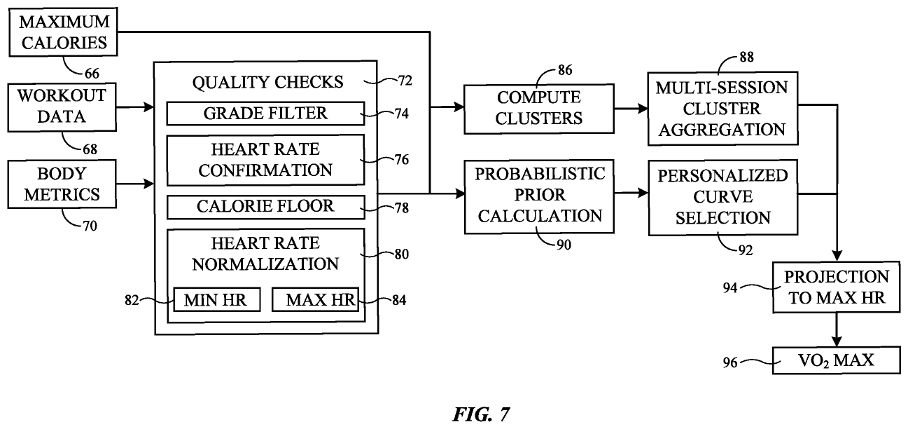

Although the steps described in connection with FIG. 6 may be useful in determining time periods that may be optimal to determine an active user's VO₂ max (e.g., to determine the VO₂ max for a user who is active at least some of the time), the method of FIG. 6 may be unable to determine a period in which a more idle user's VO₂ max may be determined. For example, step 58 of FIG. 6 requires that the user walk continuously, a characteristic that may not be possible for some users. Additionally, it may be desirable to have more personalized results (e.g., rather than extrapolating a user's heart rate based on broad data about their age and biological sex). Therefore, an additional method of determining a user's VO₂ max may be desired. An example of a system that may calculate VO₂ max values for more idle users, as well as provide more personalized results for all users, is shown in FIG. 7.

As shown in FIG. 7, a system may take maximum calories 66, workout data 68, and body metrics 70 as inputs. Maximum calories 66 may be the maximum calories burned by the user over any desired time period, such as the past week, the past month, or the past day, and may be calculated by logging and analyzing the user's heart rate and movements throughout each day and correlating the data to calories burned. Workout data 68 may include calories burned during workouts, heart rate during workouts, and any other desired workout data. In some embodiments, a user may manually indicate that a workout is being performed, engaging the requisite sensors. In other embodiments, sensors that take data occasionally may detect a spike in heart rate and movement, and automatically detect that a workout is occurring. In either case, data from the workout may be utilized in determining the user's VO₂ max. Body metrics 70 may include the user's age, physical activity level, minimum heart rate, medication status, gender, biological sex, height, weight, and any other desired factors. These body metrics may be entered manually by a user, obtained automatically through sensor measurements (e.g., such as calculating the physical activity level and minimum heart rate from measurements taken from the motion sensors and activity sensors), or obtained from doctor's through medical information exchange agreements (e.g., the user may sign up to have their medical information sent to their device for tracking purposes).

Workout data 68 and body metrics 70 may undergo quality checks 72. In particular, the data collected from the sensors within the device may be passed through grade filters 74, heart rate confirmation 76, and calorie floor 78. These filters may remove data from workout data 68 and body metrics 70 that does not meet certain criteria. For example, there may be a threshold of data that must be collected prior to being passed through a filter, data collected on graded or abnormal surfaces may be removed from the data set, the heart rate sensor may need to detect an elevated heart rate during the workouts for those workouts to be included in the data, and the user may need to burn a minimum number of calories during a workout or during a certain day for that set of data to be included. However, these quality checks are merely illustrative. In general, workout data 68 and body metrics 70 may be filtered in any desired manner to ensure quality data is used in the VO₂ max calculations.

Additionally, the user's detected heart rate may undergo heart rate normalization 80 to avoid undue influence from factors such as caffeine intake, stress, age, medication history, or any other factors. In particular, the heart rate may be normalized relative to other people and normalized relative to the user's individual baseline measurements.

A user's minimum heart rate (MIN HR 82), may be best measured from higher fidelity, more frequent heart rate sensor measurements throughout the day during periods of rest. HR MIN 82 may be selected in this way if a user begins walking from a rested state. However, if the user has an elevated heart rate at the beginning of the walk (e.g., due to stress or caffeine), HR MIN 82 may be determined by measuring the user's heart rate at the beginning of a walking period and using a logarithmic projection back to determine the minimum heart rate. For example, the logarithmic projection back may assume a first order rise in the heart rate in response to exercise. However, other back projections may be used if desired.

For some users however, there may not be sufficient heart rate sensor data to estimate HR MIN 82. Therefore, the user's HR MIN 82 may be approximated based on peak calories burned, which may in turn be estimated by the user's equivalent daily steps. In this way, a user who does not have logged heart rate data may still have a minimum heart rate determined.

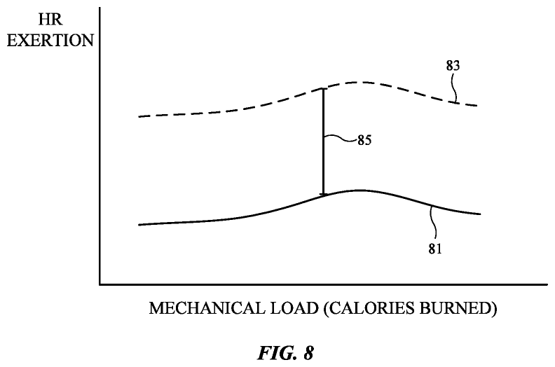

The user's maximum heart rate (MAX HR 84) may need to be modified from the heart rate measured by the heart rate sensor. For example, a lower fitness user may be on medications that affect the user's maximal heart rate, such as rate control medication or beta blockers for blood pressure control. An example of an illustrative difference in estimated MAX HR vs. actual MAX HR for a low fitness user is shown in FIG. 8.

As shown in FIG. 8, the user's estimated heart rate (based on the collected heart rate sensor data) may be given by line 81. The user's maximum heart rate may be given by line 83 yielding difference 85. In some cases, users' may be on medications that change their maximal heart rate. In other words, the user's heart rate as a fraction of their capacity may be elevated as a result of medication lowering their maximum heart rate. To correct for the use of medications, the user's HR MAX 84 may be shifted by difference 85. Although the graph of FIG. 8 has been described in connection with a user on medication, this is merely illustrative. A user's MAX HR 84 may be corrected if they have underlying health conditions such as congestive heart failure (CHF) or chronic obstructive pulmonary disease (COPD), or if they have any other underlying conditions.

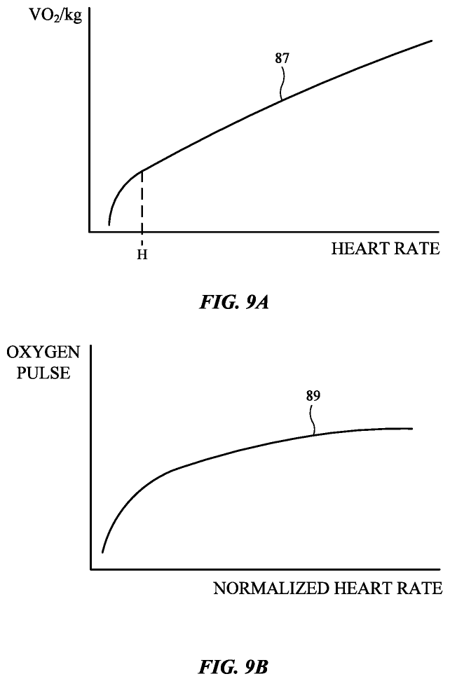

After heart rate normalization 80 has occurred, the corrected workout data 68 and body metrics 70, as well as maximum calories 66, may be used to estimate the user's VO₂ max. Although traditional models used in VO₂ max calculations may require users to exert themselves such that their heart rate is over 40%, physiological models may be used to approximate user's VO₂ max using data that is below the 40% threshold. This 40% threshold may be a measured as 40% of the user's heart rate reserve (i.e., 40% of the difference between the user's maximum heart rate and minimum heart rate). As shown in FIG. 9A, line 87 exhibits the relationship between the user's heart rate and VO₂. As shown, below heart rate H, VO₂ may have a nonlinear relationship with heart rate, while above heart rate H (such as 40%), VO₂ may be approximated with a linear relationship (e.g., FIG. 9A does not illustrate the noise in the measurements used in the relationship). A relationship between oxygen pulse space (VO₂/heart rate) vs. normalized heart rate is shown in FIG. 9B with line 89. As shown, curve 89 follows a non-linear profile for the entire relationship. Curve 89 may be approximated by any desired function, such as a quadratic curve, a polynomial curve, or a logarithmic curve (e.g., FIG. 9B does not illustrate the noise in the measurements used in the relationship. Although either relationship (e.g., the heart rate vs. VO₂ relationship of FIG. 9A or the normalized heart rate vs. oxygen pulse of FIG. 9B) may be used, the relationship of FIG. 9B is sometimes used herein as an example.

To determine a curve that correlates the individual user's heart rate to VO₂ such that the user's VO₂ max may be estimated, heart rate and physical activity data may be gathered by one or more devices and may be projected along the modeled curve to determine VO₂ max. For example, if the relation between normalized heart rate and VO₂/heart rate is logarithmic, as shown in FIG. 9B, the user's workout data and health information may be used to determine a logarithmic relationship that applies for that specific user. This may be done by computing clusters 86 of FIG. 7. In particular, clusters of the user's workout, calorie data, and body metrics may be analyzed to determine a curve that applies to the user. Specifically, a regression (or other desired statistical analysis) may be performed to determine a relationship between the user's oxygen pulse and heart rate. As a result, a curve (such a logarithmic curve) may be fit to that user's relationship between oxygen pulse and heart rate, and the user's VO₂ max may be determined from the resulting individualized oxygen pulse curve.

It may also be desired to use multi-session cluster aggregation 88 when making the determination of the user's oxygen pulse to normalized heart rate curve. In particular, by clustering multiple sessions of activity data, the effects of outlier data may be reduced.

For example, walking or running on a grade may introduce volatility into the user's heart rate and/or speed. However, using clustering techniques, steady-state portions of the user's workout may be extracted, and the data may still be used with less volatility.

In another example, many users may have insufficient data for a reliable prediction after a single walk or run workout, and user's often move at a fairly constant rate over a single session, making predictions based on a single session unreliable. However, clustering across multiple sessions may enable a single, globally optimal estimate of a user's VO₂ max. Additionally, sensors may be used outside of workouts (as described previously) to capture different ranges of walking speeds than is present in recorded workouts alone.

In another example, users may walk in undesirable terrain (such as mud, sand or snow), at altitude, carrying a heavy load, or with some other condition that may increase heart rate or reduce the user's activity output, all of which may be unobservable to the heart rate sensor and/or the motion sensors on the device. However, these scenarios are largely outlier scenarios that may be disregarded in a cluster analysis compared to other clusters from other sessions.

In another example, users may pause frequently, causing a change in heart rate, which may occur more frequently for lower fitness users. However, the cluster analysis may be at least partially removed if this is an outlier scenario (e.g., if the user is a higher fitness user), or may be included if it is not an outlier scenario. If desired, the cluster analysis may be performed with clusters of dynamic size, or the system may require that new clusters are created shortly after a pause.

In another example, large amounts of data may be excluded due to quality checks 72. However, using cluster analysis, clusters may be associated with respective confidences and weighted appropriately. Therefore, in a shortage of data, the system may keep more data in the analysis, and weight more accurate data greater than less accurate/significant data. Additionally or alternatively, more data may be gathered outside of user initiated workouts, providing more accurate data than workout-only data.

By computing clusters 86 and performing multi-session cluster aggregation 88, more accurate correlations between a user's heart rate and VO₂ may be obtained, thereby resulting in a more accurate VO₂ max estimation.

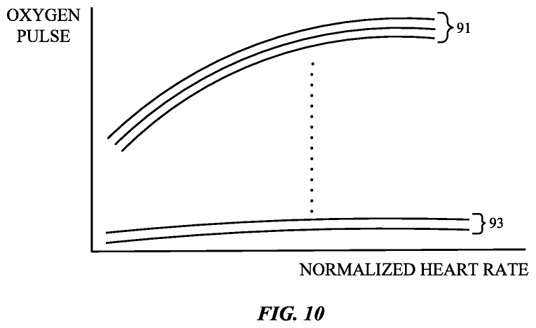

However, while the determined logarithmic relationship based on computed clusters 86 and multi-session cluster aggregation 88 may provide an accurate VO₂ max for an active user with a significant amount of workout data at high heart rates, the relationship may be different for lower fitness users. An example of this is shown in FIG. 10.

As shown in FIG. 10, high fitness users may have normalized heart rate to oxygen pulse relationships exhibited by curves 91, which may have a logarithmic (or other function) relationship. However, lower fitness users may have relationships governed by curves 93, which may have significantly less curvature than curves 91. Therefore, the same logarithmic relationship used for a high fitness user cannot merely be shifted down to account for a lower fitness user, as doing so would overestimate the user's VO₂ max. To account for users of difference fitness levels, probabilistic prior calculation 90 and personalized curve selection 92 may be performed.

Probabilistic prior calculation 90 may be based on probabilistic distributions of age, biological sex, activity (as measured by maximum calories burned over a selected time period), and fitness level (using activity information from the electronic device or calculated based on motion and activity sensor data). In particular, these factors may be used to select a predicted oxygen pulse curve shape for the user. For example, with increased age or reduced activity/fitness, a user's heart response typically becomes more blunted (e.g. the curves for those users may have a smaller slope, as illustrated by curves 93 of FIG. 10). Based on the probabilistic calculation using the user's physiological factors, a personalized curve 92 may be selected for the user.

After the cluster analysis 86/88 and personalized curve selection 92, the cluster analysis may be used to fit the personalized curve based on the aggregated cluster data. Once this correction has been made, a projection may be made to the user's maximum heart rate 94. For example, the corrected curve may be extrapolated/projected, and a maximum heart rate predicted. At this maximum heart rate, the user's VO₂ max 96 may be determined (e.g., because of the relation between the user's oxygen pulse and VO₂). By using the system shown in FIG. 7, VO₂ max values may be estimated for all users, regardless of fitness/activity level, and more accurate VO₂ max values may be obtained for all users because of the use of clustering techniques and physiological prediction techniques. In particular, VO₂ max values may be determined for users who have not exerted themselves as much as users with extensive workout data. For example, VO₂ max values may be generated for users who do not have workout data with a maximum heart rate that exhibits more than 40% of their heart rate reserve (the difference between the user's maximum heart rate and minimum heart rate), more than 50% of their heart rate reserve, or more than 60% of their heart rate reserve, as examples, as well as users who do have such heart rate data.

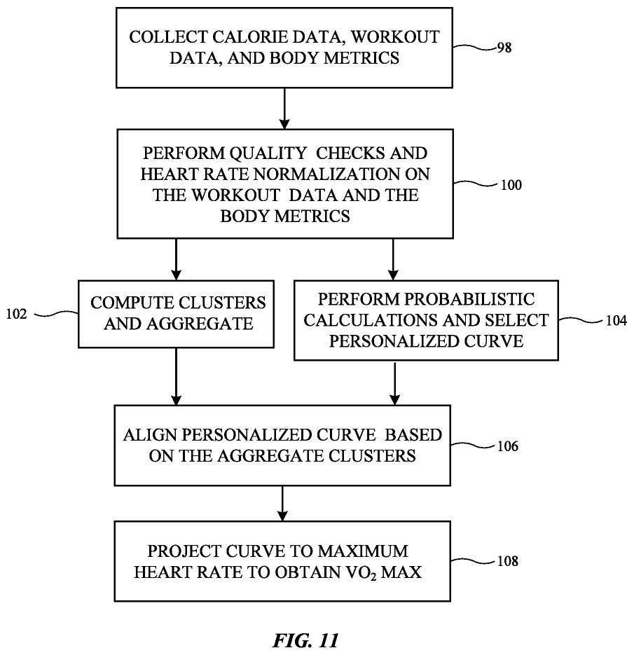

A flowchart illustrating the steps performed in connection with the diagram of FIG. 7 is shown in FIG. 11. At step 98, the control circuitry may collect calorie data, workout data, and body metric information. The calorie data may be the maximum calories burned by the user over any desired time period, such as the past week, the past month, or the past day, and may be calculated by logging and analyzing the user's heart rate and movements throughout each day and correlating the data to calories burned. The workout data may include calories burned during workouts, heart rate during workouts, and any other desired workout data. In some embodiments, a user may manually indicate that a workout is being performed, engaging the requisite sensors. In other embodiments, sensors that take data occasionally may detect a spike in heart rate and movement, and automatically detect that a workout is occurring. In either case, data from the workout may be utilized in determining the user's VO₂ max. Body metric information may include the user's age, physical activity level, minimum heart rate, medication status, gender, biological sex, height, weight, and any other desired factors. These body metrics may be entered manually by a user, obtained automatically through sensor measurements (e.g., such as calculating the physical activity level and minimum heart rate from measurements taken from the motion sensors and activity sensors), or obtained from doctor's through medical information exchange agreements (e.g., the user may sign up to have their medical information sent to their device for tracking purposes).

At step 100, the circuitry may perform quality checks and heart rate normalization of the workout data and the body metric data. In particular, the data collected from the sensors within the device may be passed through filters, heart rate confirmation, and calorie floor checks. These filters may remove data from workout data and body metric data that does not meet certain criteria. For example, there may be a threshold of data that must be collected prior to being passed through a filter, data collected on graded or abnormal surfaces may be removed from the data set, the heart rate sensor may need to detect an elevated heart rate during the workouts for those workouts to be included in the data, and the user may need to burn a minimum number of calories during a workout or during a certain day for that set of data to be included. However, these quality checks are merely illustrative. In general, the workout data and body metric data may be filtered in any desired manner to ensure quality data is used in the VO₂ max calculations.

Additionally, the user's detected heart rate may undergo heart rate normalization 80 to avoid undue influence from factors such as caffeine intake, stress, age, medication history, or any other factors. In particular, the heart rate may be normalized relative to other people and normalized relative to the user's individual baseline measurements. As described previously in connection with FIG. 7, both the user's minimum heart rate and maximum heart rate may be normalized.

At step 102, the workout data, body metric data, and calorie data may be used to compute clusters of data and aggregate the data over multiple periods. In particular, clusters of the user's workout, calorie data, and body metrics may be analyzed to determine a relationship between the user's oxygen pulse and heart rate. As a result, a curve (such a logarithmic curve) may be fit to that user's relationship between oxygen pulse and heart rate, and the user's VO₂ max may be determined from the resulting individualized oxygen pulse curve. By clustering multiple sessions of activity data, the effects of outlier data may be reduced.

At step 104, which is in parallel with step 102, probabilistic calculations may be performed, and a personalized curve may be selected for the user to estimate the relationship between the user's normalized heart rate and oxygen pulse. Probabilistic prior calculation may be based on probabilistic distributions of age, biological sex, activity (as measured by maximum calories burned over a selected time period), and fitness level (using activity information from the electronic device or calculated based on motion and activity sensor data). In particular, these factors may be used to select a predicted oxygen pulse curve shape for the user.

At step 106, the personalized curve shape determined in step 104 may be projected and/or fitted based on the cluster analysis of step 102. In this way, the personalized curve may provide a better model of the user's oxygen pulse vs. normalized heart rate.

At step 108, the projected/fitted personalized curve may be used to estimate the user's maximum heart rate and thereby estimate the user's VO₂ max (e.g., because of the relation between the user's oxygen pulse and VO₂).

After the user's VO₂ max has been estimated, it may be stored as health information. This VO₂ max value may be stored with previous estimations of VO₂ max, may be presented to a user in histograms of data, may be sent to a doctor's office, may trigger an alert to the user or to a physician, may be used by other applications on electronic device 10, electronic device 20, and/or any other desired device, or may be used in any other desired fashion.

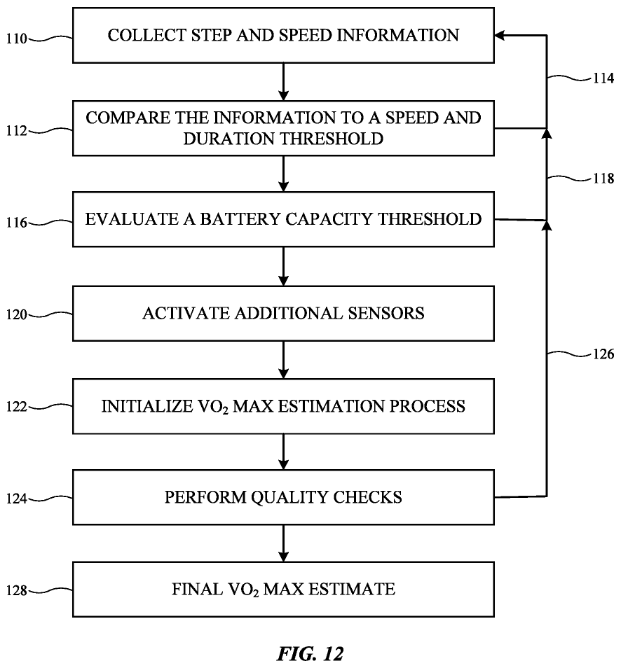

To obtain accurate VO₂ max estimates while reducing battery drain, it may be desirable to use some sensors all of the time while device 10 is in use, while only activating other sensors when needed to estimate a user's VO₂ max. An example of this is shown in FIG. 12.

As shown in FIG. 12, at step 110 step and speed information may be collected. The step and speed information may be collected by motion sensors, such as accelerometer 32 and/or gyroscope 34 of FIG. 4. These motion sensors may be collected continuously or at regular intervals while device 10 is in use.

At step 112, control circuitry may determine whether a speed and duration threshold has been met by a user of device 10. For example, the threshold may be at least 2 minutes, at least 1 minute, or any other desired duration at least 1.5 mph, at least 1.8 mph, at least 2.0 mph, or any other desired speed. If the speed and duration threshold is not met (i.e., the user has not gone a requisite speed for a minimum amount of time), the process may proceed along line 114 and continue collecting only step and speed information.

If the speed and duration threshold is met, the process may proceed to step 116, in which the control circuitry may determine whether the device has sufficient battery remaining to activate additional sensors. For example, the control circuitry may determine whether there is over 20% battery remaining, over 10% battery remaining, or any other desired battery threshold. If there is insufficient battery remaining, the process may proceed along line 118 to continue collecting step and speed information without activating additional sensors that may drain the device battery.

If there is sufficient battery, the process may proceed to step 120, in which the control circuitry may activate additional sensors that may be used to determine a user's VO₂ max. For example, the control circuitry may activate a GPS sensor, such as GPS sensor 36 of FIG. 4, a hear rate sensor, such as hear rate sensor 44 of FIG. 5, and WiFi sensors, which may be included in communications circuitry 114 of FIG. 3. However, these sensors are merely illustrative. In general, the control circuitry may activate any desired additional sensors to be used in determining the user's VO₂ max.

At step 122, the control circuitry may initialized the VO₂ max estimation process. This process may be the same or substantially the same as the VO₂ max estimation process described in connection with FIGS. 7-11. In particular, the activated sensors may be used to determine information of the user, quality checks may be performed, and statistical analyses may be used to determine the user's VO₂ max.

At step 124, quality checks may be performed, which may be the same as the quality checks discussed above at step 100 of FIG. 11. Additionally or alternatively, the control circuitry may determine whether the user has stopped walking or walked for a set amount of time (e.g., at least 5 minutes, at least 8 minutes, at least 10 minutes, or any other desired threshold). The control circuitry may also determine whether the user is walking outdoors using the device's ambient light sensors, GPS sensors, and/or any other desired sensors. If the data does not pass the quality checks (as discussed in connection with FIG. 11), or the user is indoors, the process may proceed along line 126 and the additional sensors that were activated at step 120 may be deactivated. Once the user has walked for a sufficient amount of time (i.e., over the set threshold), the data has passed the quality checks, and the user was outdoors, the process may proceed to step 128.

At step 128, the user's VO₂ max may be estimated. This estimation may be done in the same way or substantially the same way as discussed above in connection with FIGS. 7-11. After the VO₂ max has been estimated, the additional sensors that were activated at step 120 may be deactivated to save battery. In this way, the user's VO₂ max may be determined automatically based on low-intensity workout data (such as walking data), while preserving the devices battery by selectively activating and deactivating the sensors required for the VO₂ max estimation.

As described above, one aspect of the present technology is the gathering and use of information such as information from input-output devices. The present disclosure contemplates that in some instances, data may be gathered that includes personal information data that uniquely identifies or can be used to contact or locate a specific person. Such personal information data can include demographic data, location-based data, telephone numbers, email addresses, twitter ID's, home addresses, data or records relating to a user's health or level of fitness (e.g., vital signs measurements, medication information, exercise information), date of birth, username, password, biometric information, or any other identifying or personal information.

The present disclosure recognizes that the use of such personal information, in the present technology, can be used to the benefit of users. For example, the personal information data can be used to deliver targeted content that is of greater interest to the user. Accordingly, use of such personal information data enables users to calculated control of the delivered content. Further, other uses for personal information data that benefit the user are also contemplated by the present disclosure. For instance, health and fitness data may be used to provide insights into a user's general wellness, or may be used as positive feedback to individuals using technology to pursue wellness goals.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the United States, collection of or access to certain health data may be governed by federal and/or state laws, such as the Health Insurance Portability and Accountability Act (HIPAA), whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select not to provide certain types of user data. In yet another example, users can select to limit the length of time user-specific data is maintained. In addition to providing “opt in” and “opt out” options, the present disclosure contemplates providing notifications relating to the access or use of personal information. For instance, a user may be notified upon downloading an application (“app”) that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data at a city level rather than at an address level), controlling how data is stored (e.g., aggregating data across users), and/or other methods.

Therefore, although the present disclosure broadly covers use of information that may include personal information data to implement one or more various disclosed embodiments, the present disclosure also contemplates that the various embodiments can also be implemented without the need for accessing personal information data. That is, the various embodiments of the present technology are not rendered inoperable due to the lack of all or a portion of such personal information data.

The foregoing is illustrative and various modifications can be made to the described embodiments. The foregoing embodiments may be implemented individually or in any combination.

Table of Reference Numerals

| | | | |
|---|---|---|---|
| 10, 20 | Electronic Devices | 12, 22 | Housings |
| 14, 24 | Displays | 16 | Watch Band |
| 8 | System | 112, 212 | Control Circuitry |
| 114, 214 | Communications | 116, 216 | Input-Output |
| | Circuitry | | Devices |
| 118, 218 | Sensors | 122, 222 | Other Devices |
| 30 | Motion | 32 | Accelerometer |
| | Information | | |
| 34 | Gyroscope | 36 | GPS Sensor |
| 38 | Motion Sensor | 40 | Motion Metric |
| | Analysis | | Value |
| 42 | Activity | 44 | Heart Rate |
| | Information | | Sensor |
| 46 | Perspiration | 48 | Electro- |
| | Sensor | | cardiogram |
| | | | Sensor |
| 50 | Activity Sensor | 52 | Activity Metric |
| | Analysis | | Value |
| 54, 56, 58, 60, 62, 64, | Flowchart Steps | 66 | Maximum |
| 98, 100, 102, 104, | | | Calories |
| 106, 108, 110, 112, | | | |
| 114, 116, 118, 120, | | | |
| 122, 124, 126, 128 | | | |
| 68 | Workout Data | 70 | Body Metrics |
| 72 | Quality Checks | 74 | Grade Filter |
| 76 | Heart Rate | 78 | Calorie Floor |
| | Confirmation | | |
| 80 | Heart Rate | 82 | Min HR |
| | Normalization | | |
| 84 | Max HR | 86 | Compute |
| | | | Clusters |
| 88 | Multi-Session | 90 | Probabilistic |
| | Cluster | | Prior |
| | Aggregation | | Calculation |
| 92 | Personalized | 94 | Projection to |
| | Curve Selection | | Max HR |
| 96 | VO₂ Max | 81, 83, 87, 89 | Lines |
| 85 | Hear Rate | 91, 93 | Curves |
| | Difference | | |

What is claimed is:

1. An electronic device configured to be worn by a user, the electronic device comprising:
a housing;
a first sensor that measures a motion of the housing;
a second sensor that measures a heart rate of the user; and
control circuitry configured to:
generate workout data, body metric data, and calorie data based on the motion of the housing and the heart rate, and
use the workout data, body metric data, and calorie data to estimate a VO₂ max value for the user.

2. The electronic device defined in claim 1 wherein the control circuitry is configured to use the workout data, body metric data, and calorie data to estimate a VO₂ max value for the user when the maximum measured heart rate is less than 40% of a heart rate reserve of the user.

3. The electronic device defined in claim 1 wherein the control circuitry is further configured to:
normalize the heart rate of the user, and
determine a relationship between the normalized heart rate and an oxygen pulse of the user based at least in part on the workout data, the body metric data, the calorie data, and probabilistic calculations that include information on the user's age.

4. The electronic device defined in claim 3 wherein the control circuitry is further configured to estimate the VO₂ max value for the user based on the relationship between the normalized heart rate and the oxygen pulse.

5. The electronic device defined in claim 4 wherein the control circuitry is configured to determine the relationship between the normalized heart rate and the oxygen pulse based further in part on a cluster analysis of the workout data, the body metric data, and the calorie data.

6. The electronic device defined in claim 3 wherein the probabilistic calculations further include biological sex information, activity information, and fitness level information.

7. The electronic device defined in claim 6 wherein the control circuitry is configured to normalize the heart rate by normalizing the minimum heart rate of the user and the maximum heart rate of the user.

8. The electronic device defined in claim 1 wherein the control circuitry is configured to filter the workout data and the body metric data based on the measured heart rate and the measured motion of the housing.

9. The electronic device defined in claim 1 wherein the first sensor is an accelerometer and wherein the electronic device further comprises:
a global positioning system sensor that measures additional aspects of the motion of the housing, wherein the control circuitry is configured to analyze data from the accelerometer and the global positioning system sensor to generate the workout data, the body metric data, and the calorie data.

10. The electronic device defined in claim 9 wherein the second sensor is a photoplethysmography sensor, wherein the electronic device further comprises an electrocardiogram sensor, and wherein the control circuitry is configured to determine the workout data, the body metric data, and the calorie data based on data from the photoplethysmography sensor and data from the electrocardiogram sensor.

11. A method of estimating a VO₂ max value for a user of an electronic device having a motion sensor and an activity sensor, the method comprising:
gathering motion data using a motion sensor;
gathering heart rate data using a heart rate sensor;
generating calorie data, workout data, and body metric data based at least in part on the motion data and the heart rate data;
normalizing the heart rate data;
generating a personalized relationship between the normalized heart rate data and an oxygen pulse based on the calorie data, the workout data, and the body metric data; and
estimating the VO₂ max value based on the personalized relationship between the normalized heart rate data and the oxygen pulse.

12. The method defined in claim 11 generating the personalized relationship between the normalized heart rate data and an oxygen pulse further comprises generating the personalized relationship based on clustered data from multiple time periods and probabilistic prior calculations.

13. The method defined in claim 12 wherein generating the personalized relationship based on the probabilistic prior calculations comprises generating the personalized relationship based on at least a given one of the user's age, biological sex, and fitness level.

14. The method defined in claim 12 further comprising:
filtering the workout data and the body metric data based on the heart rate data and the motion data.

15. The method defined in claim 11 wherein gathering the motion data using the motion sensor comprises generating the motion data using at least one sensor selected from the group consisting of: an accelerometer, a gyroscope, and a pressure sensor, and wherein gathering the heart rate data using a heart rate sensor comprises using a photoplethysmography sensor.

16. The method defined in claim 15 wherein gathering the motion data further comprises using a global positioning system sensor.

17. An electronic device configured to be worn by a user, the electronic device comprising:
a housing;
a motion sensor in the housing;
a heart rate sensor in the housing; and
control circuitry configured to:
determine that the user is at their maximal ability based on data from the motion sensor and data from the heart rate sensor,
predict that the user will walk for an uninterrupted period of time,
take varied intensity profiles of the user's motion and heart rate during the uninterrupted period of time, and
calculate a VO₂ max value based on the varied intensity profiles.

18. The electronic device defined in claim 17 wherein the control circuitry is further configured to:
take additional varied intensity profiles at randomized times throughout the day, wherein the VO₂ max value is based on the varied intensity profiles and the additional varied intensity profiles.

19. The electronic device defined in claim 17 wherein the control circuitry is configured to take the varied intensity profiles only when the user is predicted to walk for an uninterrupted period of time.

20. The electronic device defined in claim 19 wherein the control circuitry is configured to operate the motion sensor and the heart rate sensor at an increased frequency when taking the varied intensity profiles.

21. The electronic device defined in claim 17 wherein the housing is configured to be worn on the user's wrist, wherein the motion sensor is an accelerometer, and wherein the heart rate sensor is a photoplethysmography sensor.

22. The electronic device defined in claim 17 wherein the control circuitry is further configured to:
collect step and speed information using the motion sensor; and
selectively activate the heart rate sensor and additional sensors when the step and speed information exceeds a threshold.

23. The electronic device defined in claim 22 wherein the step and speed information threshold is at least 1.8 mph for at least 2 minutes.
